(19) United States
(12) Patent Application Publication
(10) Pub. No.: US 2011/0200708 A1
(43) Pub. Date: Aug. 18, 2011

(54) A COMPOSITION FOR ACTIVATING AND/OR STABILIZING MICRO-ORGANISMS

(76) Inventors: Brian J. Orme, Longan/River Heights, UT (US); Randalf Kirk Thunell, Logan, UT (US); Gary K Burningham, Logan, UT (US); Hendrik Louis Bijl, Vlaardingen (NL)

(21) Appl. No.: 13/126,250

(22) PCT Filed: Nov. 2, 2009
(86) PCT No.: PCT/EP2009/064428

§ 371 Date: Apr. 27, 2011

Related U.S. Application Data
(60) Provisional application No. 61/110,148, filed on Oct. 31, 2008.

(30) Foreign Application Priority Data
Mar. 2, 2009 (EP) 09154139.1

(51) Int. Cl. C12N 1/20; A23C 9/12

(52) U.S. Cl. 426/42; 435/253.6; 435/252.1

(57) ABSTRACT

The present invention is in the field of production of fermented products. More in particular, it relates to improved compositions for making dairy products, in particular improved bulk starter inoculum compositions and improved bulk starters.

### FIELD OF THE INVENTION

The present invention is in the field of production of fermented products. More in particular, it relates to improved compositions for making dairy products, in particular improved bulk starter inoculum compositions and improved bulk starters.

### BACKGROUND OF THE INVENTION

Micro-organisms are involved in the production of many food and feed products including fermented dairy products such as cheese, yoghurts, sour cream, kefir, butter and koumiss. Selected strains of micro-organisms initiating and carrying out the desired fermentation essential in the manufacture of the above products are often referred to as starter cultures. In particular, cultures of lactic acid bacteria are widely used as starter cultures.

Lactic acid bacteria commonly used in the food industry can be divided into mesophilic lactic acid bacteria including the genera Lactococcus, Leuconostoc and Pediococcus and thermophilic lactic acid bacteria including the genera Streptococcus and Lactobacillus. While mesophilic lactic acid bacteria have a optimum growth temperature of about 30° C., thermophilic lactic acid bacteria have a optimum growth temperature of about 40° C. to about 45° C.

Starter cultures are generally available from commercial manufacturers in lyophilized, frozen or liquid form. They can comprise only a single lactic acid bacterium species, but can also be mixed cultures comprising two or more different lactic acid bacterium species. Mixed starter cultures are often used to minimize the negative consequences of bacteriophage infection.

Starter cultures can be inoculated directly into milk without intermediate transfer and/or propagation. Such starter cultures are generally referred to as direct vat set (DVS) or direct to vat inoculation (DVI) cultures. Despite the availability of DVS and DVI cultures, it is not uncommon that dairies produce in-house bulk starter cultures. Bulk starter cultures are made by inoculating a growth medium using a small amount of a starter culture followed by incubating the growth medium under conditions permitting the bacteria to propagate for a sufficient period of time to provide desired cell numbers and activity. The obtained bulk starter culture is then used to inoculate milk for the manufacture of dairy products.

Acidity is one of the major control factors in the making of cheese and other dairy products and is developed by adding a starter culture of lactic acid bacteria to milk. Acid production is the major function of these starter bacteria. The bacteria ferment lactose present in the milk to produce lactic acid. Failure of the milk and the starter culture to develop acidity at a sufficient rate can result in allowing harmful or undesirable microorganisms to grow. It can also result in flavour taint, high moisture and a weak curd in the cheese. Moreover, acid development contributes to proteolysis and flavour production in cheese and affects rheological properties of cheese. Too much acidity developing too quickly can result in cheese with poor body and texture. The correct development of controlled acidity is therefore critical to the good manufacture of cheese and to inhibit the proliferation of harmful and spoilage microorganisms.

It has been observed that cells of starter cultures have a reduced viability and consequently rapidly loose their metabolic activity, such as e.g. their acidification activity, when they are subjected to stress during harvesting, packaging and storage even for short periods of time. Stress can for instance be caused by storage under low temperatures (cold stress). It is known that the viability of starter cultures can be preserved by e.g. the addition of cryoprotectants (see e.g. U.S. Pat. No. 3,897,307 and EP 0 259 739).

WO 2006/067136 describes a starter culture composition comprising 20 to 80% (dry matter) of a stimulant, preferably the stimulant is a yeast extract or protein hydrolysate, and 80 to 20 wt % of starter culture. The starter culture composition may further comprise culture growth factors, preferably the growth factors are minerals or vitamins. The composition may also comprise stabilizing agent(s) and/or buffering agent(s).

In view of the importance of the performance of starter cultures, especially with respect to their metabolic activity, there is a continuing need in the art to provide starter cultures having improved storage stability. The present invention addresses this need.

### DESCRIPTION OF THE FIGURES

FIG. 1: Temperature versus time of a culture concentrate formulated in a composition containing 32% glycerol or a composition according to the invention in a study of a cooling/freezing process in a −70° C. freezer. The lower line is the culture concentrate formulated in the composition according to the invention and the upper line is the culture concentrate formulated in the composition comprising 32% glycerol.

### SUMMARY OF THE INVENTION

The present inventors surprisingly established that the combined presence of yeast extract, a polysaccharide, and a cryoprotectant has a very positive effect on the stability and/or activity of micro-organisms used in the production of dairy products. The compounds in the compositions according to the invention potentiate the action of each other, so that the combination provides a product wherein micro-organisms show good cell viability both during the rigors of a cooling process and upon storage.

### DETAILED DESCRIPTION OF THE INVENTION

The present invention provides a micro-organism activating and/or a micro-organism stabilising composition comprising:
yeast extract,
a polysaccharide, and
a cryoprotectant.

The term “micro-organism”, as used herein, typically refers to a micro-organism used in the production of fermented dairy products, such as, but not limited to, a lactic acid bacterium. “Lactic acid bacteria” as used herein refers to Gram-positive, low-GC-content, acid tolerant, generally non-sporulating, non-respiring rods or cocci that are associated by their common metabolic and physiological characteristics. They ferment carbohydrates to produce acids, including lactic acid as the predominantly produced metabolic end product. They constitute a heterogeneous group including, but not limited to, the genera Aerococcus, Carnobacterium, Enterococcus, Lactobacillus, Lactococcus, Leuconostoc, Oenococcus, Pediococcus, Sporolactobacillus, Streptococcus, Tetragenococcus, Vagococcus, and Weisella.

Alternatively, the invention provides a starter culture activating and/or a starter culture stabilising composition comprising
yeast extract,
a polysaccharide, and
a cryoprotectant.

“Starter culture” as used herein refers to a preparation containing microbial cells that is intended for inoculating a medium to be fermented. “Bulk starter culture” as used herein means a starter culture propagated at a dairy plant for inoculation into a substrate, e.g. milk.

Starter cultures, such as bulk starter cultures, may comprise one lactic acid bacterium strain or several different lactic acid bacteria strains. Besides that, a (bulk) starter culture may comprise other bacterial strains including, but not limited to, strains of the genera Bifidobacterium, Brevibacterium and Propionibacterium. Besides the above bacterial strains other microbial strains such as strains of moulds, fungi and/or yeasts including, but not limited to, the genera Penicillium, Geotrichum, Saccharomyces and Kluyveromyces may be included in a (bulk) starter culture.

Lactic acid bacteria can be divided into mesophilic and thermophilic lactic acid bacteria. Lactococcus, Leuconostoc and Pediococcus are examples of genera belonging to the mesophilic lactic acid bacteria. Examples of genera belonging to the thermophilic lactic acid bacteria are Streptococcus and Lactobacillus.

An example of a Lactococcus species is Lactococcus lactis. This species can be further subdivided into subspecies including, but not limited to, Lactococcus lactis cremoris, Lactococcus lactis lactis, Lactococcus lactis lactis biovar. diacetylactis and Lactococcus lactis hordniae. Examples of Leuconostoc species are Leuconostoc mesenteroides cremoris and Leuconostoc paramesenteroides. Examples of Pediococcus species are Pediococcus adidilacti and Pediococcus pentosaceus. Examples of Streptococcus are Streptococcus thermophilus and Streptococcus salivarius thermophilus. Examples of Lactobacillus species are Lactobacillus helveticus, Lactobacillus delbruekii ssp. bulgaricus, Lactobacillus casei and Lactobacillus acidophilus.

The term “activating” refers to the activity of the used micro-organism in a suitable medium and typically refers to the acidification activity or growth rate of said micro-organism. One example of a suitable medium is milk and in that case the “acidification activity” as used herein means a change (i.e. decrease) in pH of milk in time when inoculated with a culture formulated in a composition according to the invention and for example compared to a culture having approximately the same cell numbers, but which is not formulated in a composition according to the invention. A composition is considered to be a micro-organism activating composition if the decrease in pH (ΔpH/time period) is larger when compared to the decrease of pH (ΔpH/time period) of a control composition. The control composition comprises the same type of micro-organism, preferably with the same cell numbers, but the micro-organisms are not formulated in a composition according to the invention. In the control composition the micro-organisms are either not formulated or formulated in a composition that differs from the composition according to the invention.

The term “stabilising” refers to the effect of storage on the activity of a micro-organism formulated in a composition according to the invention. More specific, the stability of a micro-organism stored in a composition according to the invention does not or hardly not change, i.e. upon inoculation of a micro-organism (formulated in a composition according to the invention) the same or almost the same activity (ΔpH/time period) is found as for a non-stored, just formulated micro-organism. In an embodiment the change of activity between the micro-organism stored in the composition according to the invention and a freshly prepared micro-organism is 0.50 (ΔpH/time period) or less, preferably 0.25 (ΔpH/time period) or less, more preferably 0.10 (ΔpH/time period) or less. In an embodiment it is within the range of 0-0.5, preferably 0-0.25, more preferably 0-0.10 (ΔpH/time period). In another embodiment it is within the range of 0.05-0.5, preferably 0.05-0.25, more preferably 0.05-0.10 (ΔpH/time period). In a preferred embodiment there is no or hardly any change in activity. In an embodiment the storage of the micro-organism (e.g. in the form of a starter culture) takes place for a period of 1 to 12 months, for example 1, 2, 3, 4, 5, 6, 7, 8, 9, 10, 11, 12, 18 or 24 months. In an embodiment a micro-organism stored in a composition according to the invention for at least 1 months, 2 months, 3 months, 4 months, 5 months, 6 months, 7 months, 8 months, 9 months, 10 months, 11 months, 12 months, 18 months and preferably at least 24 months still has at least 80%, preferably at least 85%, more preferably at least 90%, even more preferably at least 95%, in particular at least 97%, more in particular at least 98% and most in particular at least 99% of the acidification activity of a freshly prepared micro-organism. In an embodiment the used storage condition is from −80 to −20° C. or −60 to −20° C. or −50 to −20° C. or −40 to −20° C. or −30 to −20° C. In a preferred embodiment, a composition according to the invention is at least capable of activating a micro-organism. In yet another preferred embodiment, a composition according to the invention stabilizes a micro-organism and in yet another preferred embodiment, a composition according to the invention is capable of activating and stabilizing a micro-organism (e.g. in the form of a starter culture).

The yeast extract used in a composition according to the invention can be obtained from all kinds of different yeast species. Preferably, the yeast extracts of Saccharomyces spp., Kluyvermomycesa spp., or Torula spp. are used. Without being bound by it, the inventors of the current invention believe that the yeast extracts act as a stimulant by having a positive effect on the acidification activity of the used micro-organism. More preferably, an extract of Saccharomyces spp., such as S. cerevisiae is used. Examples of suitable yeast extracts are liquid Gistex®, Amberferm 5001® yeast extract powder or HiYest®.

The yeast extract can be a liquid or a solid yeast extract (i.e. a powder) or a combination thereof. The liquid yeast extract may be an aqueous yeast extract. The invention therefore provides a micro-organism activating and/or a micro-organism stabilising composition comprising:
yeast extract,
a polysaccharide, and
a cryoprotectant,
wherein said yeast extract is a liquid yeast extract, a solid yeast extract or a combination thereof.

In an embodiment the used yeast extract is essentially solid yeast extract (i.e. in the absence of liquid yeast extract), such as yeast extract powder. The yeast extract powder not only has a positive effect on the activity of a micro-organism, but also contributes to a reduction in the water activity due its water binding capacity.

Another ingredient of a composition according to the invention is a polysaccharide. The function of the polysaccharide is to increase the viscosity of the composition according to the invention. As a result, the composition of the invention does not freeze or only partly freezes at low temperatures such as e.g. temperatures of −60 to −20° C. Examples of suitable polysaccharides are cellulose, starch, (Arabic) gum, micro-crystalline cellulose (MCC), modified starch, guar gum or xanthan gum. Moreover, any of these polysaccharides can be combined. Preferably, the used polysaccharide is cellulose such as, but not limited to, Solka-Floc (for example Solka-Floc 300 FCC). Instead of a polysaccharide or next to a polysaccharide, a composition according to the invention can also comprise a polypeptide. The polysaccharide may be present in the composition according to the invention as a liquid (e.g. a solution comprising polysaccharide) or a solid.

Yet another ingredient of a composition according to the invention is a cryoprotectant. Suitable examples of a cryoprotectant are polyalcohols such as glycerol, mannitol, sorbitol and propylene glycol; sugars such as sucrose, fructose, lactose, dextrose, and trehalose; polyethers such as polypropylene glycol or polyethylene glycol; protein hydrolysates; amino acids like monosodium glutamate or any combination thereof. The cryoprotectant mat be present in the composition according to the invention as a solid or a liquid (e.g. an aqueous cryoprotectant solution).

The inventors of the herein described invention have noticed that the above-described composition reduces ice crystal formation. Micro-organisms in a composition according to the invention are therefore not damaged (or less damaged) at lower temperatures and consequently the viability of the micro-organisms is increased.

Moreover, a composition according to the invention with added micro-organisms (e.g. a starter culture composition comprising a composition according to the invention and micro-organisms) has a flow-out at −20 to −45° C. from a 3-5 liter bag of 5 minutes or less. Such micro-organisms (e.g. in the form of a starter culture) can thus be added in a very convenient and fast manner to for example a cheese vat or bulk starter vessel.

As described above, the invention provides a micro-organism activating and/or a micro-organism stabilising composition comprising
yeast extract,
a polysaccharide, and
a cryoprotectant.

It will be clear to the skilled person that multiple other components can advantageously be added to a composition according to the invention, i.e. a composition according to the invention typically further comprises at least one additional compound.

In an embodiment the composition according to the invention further comprises iron, manganese, molybdenum or zinc salts or any combination thereof. The presence of any of these metals (or a combination of any of them) may act as a stimulant by having a positive effect on the acidification activity of the used micro-organism. The iron salt can be a Fe²⁺ (ferrous) or a Fe³⁺ (ferric) salt. In general, ferric salts tend to have a very low solubility. Preferably, the used iron salt is therefore a ferrous salt. As disclosed herein, suitable examples of iron slats are iron sulphate (i.e. Fe being Fe²⁺), iron pyrophosphate (i.e. Fe being Fe³⁺), iron gluconate (i.e. Fe being Fe²⁺) or iron citrate (i.e. Fe being Fe²⁺). As shown herein, the addition of iron salts leads to improved acidification activity. The present inventors have however noticed that, especially upon large scale preparation, the iron sulphate tends to precipitate. Alternatively, iron gluconate (which does not have the problem of precipitation) can be used in a composition according to the invention. The invention therefore provides a micro-organism activating and/or a micro-organism stabilising composition comprising:
yeast extract,
iron (e.g. iron gluconate), manganese, molybdenum or zinc salts or any combination thereof,
a polysaccharide, and
a cryoprotectant.

Manganese can be provided as manganese sulphate, as manganese chloride or as any other salt. In an embodiment the composition according to the invention comprises an iron salt and a manganese salt. Said iron salt may be a ferrous salt. Molybdenum can for example be provided as Na₂MoO₄.H₂O. As disclosed herein within the experimental part, molybdenum provides stimulation of acidification activity on its own as well as in combination with a yeast extract and/or iron and manganese. Zinc can for example be provided as zinc sulphate.

A further example of an additional compound is a de-foamer. The invention therefore also provides a micro-organism activating and/or a micro-organism stabilising composition according to the invention comprising a de-foamer.

Suitable examples of a defoamer are a silicium de-foamer suspension, Magrabal Silicone, SAG, Basildon or other de-foamers like Clerol, Structol and or mixtures with vegetable oils (such as soy oil). Of course, combinations of suitable de-foamers can also be used.

Another example of an additional compound is a pH regulator or a pH (titrant) controller. The invention therefore also provides a micro-organism activating and/or a micro-organism stabilising composition according to the invention comprising a pH regulator or a pH (titrant) controller.

Preferably, said pH regulator or pH (titrant) controller is a compound or agent used to set the pH. A suitable pH regulator is an ammonium hydroxide solution, a potassium hydroxide solution or a sodium hydroxide solution, to name just a few.

Further suitable additional compounds include, but are not limited to, a growth stimulating agent or a stabilising agent or any mixture thereof.

Growth stimulating agents include, but are not limited to, carbohydrates such as sugars, vitamins, protein hydrolysates, minerals, proteins, peptides, nucleosides, nucleobases, amino acids, nucleotides, porphyrins and mixtures thereof.

Stabilising agents include, but are not limited to, formic acid, formate, inosinate, inosine, natural and/or chemical antioxidants, nucleosides, nucleobases, amino acids, nucleotides, surfactants such as Tween compounds, hypoxanthine, xanthine and mixtures thereof.

A composition according to the invention can comprise at least one, at least two or even more than two additional compounds. In a preferred embodiment, a composition according to the invention comprises two additional compounds, for example a de-foamer and a pH regulator, i.e. the invention also provides a micro-organism activating and/or a micro-organism stabilising composition comprising:
yeast extract,
a polysaccharide,
a cryoprotectant,
a de-foamer, and
a pH regulator

Optionally, the composition according to the invention also comprises iron, manganese, molybdenum or zinc salts or any combination thereof.

In one of its aspect, the invention provides a composition as described above comprising:
5-45 wt % yeast extract,
0.5-2.5 wt % polysaccharide,
20-80 wt % cryoprotectant,
wherein the total percentage of the compounds is 100. Unless stated otherwise the used percentages are weight percentages (wt %). In an embodiment the composition according to the invention comprises 5-45 wt %, preferably 10-40 wt % and more preferably 15-35 wt % yeast extract. In a further embodiment the composition according to the invention comprises 0.5-2.5 wt %, preferably 0.7-2.0 wt % and more preferably 1.0-1.5 wt % polysaccharide. In yet a further embodiment the composition according to the invention comprises 20-80 wt %, preferably 30-78 wt % and more preferably 50-75 wt % cryoprotectant.

It is clear to the skilled person that if any additional compound is used in a composition according to the invention, their amount is also included, for example the invention also provides a composition comprising:
5-45 wt % yeast extract,
0.1-10 wt % iron, manganese, molybdenum or zinc salts or any combination thereof,
0.5-2.5 wt % polysaccharide,
20-80 wt % cryoprotetcant, and
0.05-2 wt % de-foamer,
wherein the total percentage of the compounds is 100. In an embodiment the composition according to the invention further comprises 0.1-10 wt %, preferably 0.3-8 wt % and more preferably 0.5-5 wt % iron, manganese, molybdenum or zinc salts or any combination thereof. In a further embodiment the composition according to the invention comprises 0.05-2 wt %, preferably 0.1-1.5 wt % and more preferably 0.2-1 wt % de-foamer.

When a pH regulator is used as well, the percentages are for example:
5-45 wt % yeast extract,
0.1-10 wt % iron, manganese, molybdenum or zinc salts or any combination thereof,
0.5-2.5 wt % polysaccharide,
20-80 wt % cryoprotectant or polyalcohol or a combination thereof,
0.05-2 wt % de-foamer,
0.05-3 wt % pH regulator,
wherein the total percentage of the compounds is 100. In an embodiment the composition according to the invention further comprises 0.05-3 wt %, preferably 0.1-2.5 wt % and more preferably 0.5-2 wt % pH regulator.

The experimental part describes multiple examples of suitable compositions. One particular useful example is a composition comprising:

| | |
|---|---|
| liquid yeast extract (50%) | 22.5 wt % |
| ferrous gluconate powder | 1.0 wt % |
| 10% Si de-foamer suspension | 0.3 wt % |
| Yeast extract powder | 7.0 wt % |
| Cellulose (Solka-Floc 300 FCC) | 1.2 wt % |
| Ammonium hydroxide solution (29%) | 1.0 wt % |
| Glycerol | 67.0 wt % |

The components of the compositions according to the invention are mixed and neutralized to pH 6.0±0.1 pH unit before sterilization. The obtained composition is preferably cooled before addition to a micro-organism. Preferably, the composition is as cold as possible (but above 0° C.) when added to a culture concentrate (i.e. a concentrate of micro-organisms). The cooled composition is gradually added (e.g. pumped) into a cold culture concentrate under stirring. The total mixture (i.e. a composition according to the invention including a culture concentrate) is well but gently mixed to avoid introduction of air in the mixture. The mixture is subsequently allowed some time to age (preferably approximately 30 minutes) for moisture equilibration. Finally, the mixture is dispersed into a packaging material, for example a bag and stored at a temperature between e.g. −80 to −20° C.

One of the advantages of a composition according to the invention (with or without added cell culture concentrate) is that it remains liquid (or for the larger part liquid) at low temperatures such as −40 to −60° C. For example, the experimentally described above composition was mixed as described above with a cell culture concentrate and subsequently stored at −45° C. The formulated cell culture concentrate was transferred to −20° C. and stored at this temperature for 8 hours. Next, the bag comprising said formulated cell culture concentrate was dosed into a starter tank. Surprisingly, this dosing took only about 3 to 5 minutes which is extremely fast.

The invention therefore also provides a micro-organism activating and/or a micro-organism stabilising composition comprising:
yeast extract,
a polysaccharide,
a cryoprotectant,
which is liquid or semi-liquid at a temperature of −60 to −20° C.

More preferably, said composition is liquid in the range of −60 to −30, −60 to −40, −50 to −20, −40 to −20, or −30 to −20° C. The composition may of course also comprise one or more additional components as described above.

A composition according to the invention is typically used for formulating micro-organisms, preferably lactic acid bacteria. The composition and the micro-organisms are added together and when necessary, treated to obtain a homogenous mixture. The mixture can be prepared directly in a packing material or the mixture can be prepared first and later on packaged. After packaging the resulting mixture is typically stored at temperatures below −20, −30, −40, −50, −60 or below −70° C., such as but not limited to −80° C. In general, the storage temperature is between −80 to −20° C. Preferred storage conditions are −60 to −30, −60 to −40, −50 to −20, −45 to −20, −40 to −20, −35 to −20, or −30 to −20° C. These kinds of temperatures are used for transport as well. However, if transport is being performed on dry ice, the temperature might even drop to −80° C.

In another aspect the invention therefore provides a method for preparing a formulated culture concentrate comprising combining a composition as described above with a culture concentrate, optionally mixing the compounds and/or packaging the formulated culture concentrate and/or lowering the temperature to −80° C. to −20° C.

The ratio of the composition versus the culture concentrate can vary from 1:1 to 6:1 and depends for example on the type of bacteria and the culture concentrate (for example the amount of micro-organisms in said culture concentrate). In a preferred embodiment, the invention provides a method for preparing a formulated culture concentrate comprising combining a composition as described above with a culture concentrate, optionally mixing the compounds and/or packaging the formulated culture concentrate and/or lowering the temperature to −60 to −20° C., wherein 1 to 6 parts of a composition as described above and 1 part of a culture concentrate are used or alternatively worded, wherein the ratio of composition and culture concentrate is 1:1 to 6:1. A typical ratio is 3:1, i.e. 3 parts of a composition according to the invention and 1 part of micro-organisms (typically a cell culture concentrate).

The obtained mixture of a composition according to the invention and a culture concentrate typically comprises a content of viable cells of at least 1×10⁷ cfu/ml, preferably at least 1×10⁸ cfu/ml, more preferably at least 1×10⁹ cfu/ml, even more preferably at least 1×10¹⁰ cfu/ml, yet even more preferably at least 1×10¹¹ cfu/ml, in particular at least 1×10¹² cfu/ml and more in particular at least 1×10¹³ cfu/ml. Preferably, the viable cells are cells such as lactic acid bacteria.

As already described, the obtained mixtures of a composition according to the invention and a culture concentrate are (preferably aseptically) packed in suitable bags (laminate).

The obtained mixture of a composition according to the invention and a culture concentrate is preferably liquid at a temperature of −60 to −20° C. This liquid characteristic can be defined based on the flow from, for example, a bag. After storage for a certain amount of time at −78° C. or −60 to −30° C. the bag is transferred to −20° C. Typically, after an 8 hours storage at −20° C. a 4-5 kg bag is emptied within 3-5 minutes. The invention thus provides a method for preparing a formulated culture concentrate comprising combining a composition as described above with a culture concentrate, optionally mixing the compounds and/or packaging the formulated culture concentrate and/or lowering the temperature to −60 to −20° C., wherein the formulated culture is liquid at −60 to −20° C. Other preferred temperatures are −50, −45, −40, −35 and −30° C.

Even more preferred, the invention provides a method for preparing a formulated culture concentrate comprising combining a composition as described above with a culture concentrate, optionally mixing the compounds and/or packaging the formulated culture concentrate and/or lowering the temperature to −80 to −20° C., wherein said culture concentrate comprises lactic acid bacteria. Suitable examples of lactic acid bacteria have been described herein before.

The invention further provides formulated micro-organisms obtained by a method for preparing a formulated culture concentrate comprising combining a composition as described above with a culture concentrate, optionally mixing the compounds and/or packaging the formulated culture concentrate and/or lowering the temperature to −80 to −20° C. Such micro-organisms are characterised by having a good activation and/or a good stability and/or a good flow ability.

Alternatively worded, the invention provides a formulated culture concentrate comprising a composition as described herein before and a (starter) culture concentrate.

The invention further provides a formulated culture concentrate comprising
2.5-39 wt % yeast extract,
0.25-2.2 wt % polysaccharide,
10-70 wt % cryoprotecant, and
15-70 wt % culture concentrate,
wherein the total percentage of the compounds is 100.

In an embodiment the formulated culture concentrate according to the invention comprises 2.5-39 wt %, preferably 5-35 wt % and more preferably 10-30 wt % yeast extract. In a further embodiment the formulated culture concentrate according to the invention comprises 0.25-2.2 wt %, preferably 0.4-2 wt % and more preferably 0.5-1.5 wt % polysaccharide. In yet a further embodiment the formulated culture concentrate according to the invention comprises 10-70 wt %, preferably 20-65 wt % and more preferably 30-60 wt % cryoprotectant. In yet a further embodiment the formulated culture concentrate according to the invention comprises 15-70 wt %, preferably 20-65 wt % and more preferably 25-60 wt %. The ranges as mentioned here depend on the ratio used for mixing a composition according to the invention versus the amount of used culture concentrate.

When a composition according to the invention further comprises iron, manganese, molybdenum or zinc salts or any combination thereof the final amount thereof is 0.05-8.5 wt %, preferably 0.1-6 wt % and more preferably 0.2-4 wt %. When a composition according to the invention further comprises a de-foamer, the final amount of de-foamer in a formulated culture concentrate is typically 0.025-1.8 wt %, preferably 0.03-1.4 wt % and more preferably 0.05-1 wt %. For an optional pH regulator the final amount is typically 0.025-2.6 wt %, preferably 0.1-1.5 wt % and more preferably 0.3-1 wt %.

The obtained formulated culture concentrate is typically liquid at a temperature of −60 to −20° C., for example between −60 and −30° C. As a result, such a formulated culture concentrate can be added very fast to, for example, a cheese vat or bulk starter vessel.

As described in more detail in the experimental part herein, a composition as disclosed herein, i.e. a composition comprising:
yeast extract,
a polysaccharide, and
a cryoprotectant
   is capable of activating and/or stabilizing a micro-organism. The composition may comprise one or more additional components as described above.

The invention therefore also provides use of a composition comprising:
yeast extract,
a polysaccharide, and
a cryoprotectant
   for activating and/or stabilizing a micro-organism.

Alternatively, the invention provides a method for activating and/or stabilizing a micro-organism comprising adding to said micro-organism a composition comprising:
yeast extract,
a polysaccharide, and
a cryoprotectant.

Preferably, said micro-organism is a lactic acid bacterium. Suitable examples of lactic acid bacteria have already been described herein. Even more preferably, said micro-organism is a starter culture. The micro-organism used can be a themophilic lactic acid bacterium or a mesophilic lactic acid bacterium. However, different micro-organisms can also be combined, for example 2 or 3 different thermophilic lactic acid bacteria can be formulated in one bag. Mesophilic lactic acid bacteria are typically kept separate, i.e. one bag comprises a mesophilic lactic acid bacterium and a second bag comprises a different mesophilic lactic acid bacterium.

A formulated culture concentrate can be used as a starter culture directly into a suitable substrate (e.g. milk) for producing a fermented dairy product without intermediate transfer and/or propagation. Such a culture is generally referred to as direct vat set (DVS) or direct to vat inoculation (DVI) culture. However, it is not uncommon that dairies produce in-house bulk starter cultures. Bulk starter cultures are made by inoculating a growth medium using a small amount of a starter culture followed by incubating the growth medium under conditions permitting the bacteria to propagate for a sufficient period of time to provide desired cell numbers and activity. The obtained bulk starter culture is then used to inoculate a suitable substrate (e.g. milk) for the manufacture of a fermented dairy product (e.g. cheese).

In yet another embodiment, the invention provides a method for preparing a bulk starter culture comprising adding a formulated culture concentrate as described above to a suitable medium and propagating the culture to a desired density. The bulk starter culture prepared may be a mixed bulk starter culture comprising mesophilic and/or thermophilic lactic acid bacteria. Mixed cultures can be made by introducing several bags, each bag comprising a different strain into a bulk starter tank or vessel or by introducing one bag comprising different lactic acid bacteria strains into a bulk starter tank or vessel. For instance a mixed bulk starter can be made by introducing the content of a bag comprising a Lactococcus lactis subsp. cremoris strain and the content of a bag comprising a Lactococcus lactis subsp. lactis strain into a bulk starter tank or vessel. Optionally, the content of a bag comprising a Streptococcus salivarius thermophilus strain can also be introduced into the bulk starter tank or vessel.

Incubation is done at a temperature conducive to the growth of the microbial starter organisms for a period of time until the desired cell concentration and activity of the bulk starter culture are reached. Incubation can be slowed down by cooling to e.g. below 10° C. In the introduction step the growth medium is inoculated with a formulated culture concentrate according to the invention and in the incubation step the micro-organisms in said formulated culture concentrate are grown/propagated in the growth medium. In other words, in the introduction step an inoculated medium is produced and in the incubation step the inoculated medium in ripened to produce a bulk starter culture.

In an embodiment the growth medium is incubated for less than ten hours, preferably less than nine hours, more preferably less than eight hours, and in particular less than seven hours. Preferably, the incubation period is between 1 and 10 hours, more preferably between 2 and 9 hours, even more preferably between 3 and 8 hours and most preferably between 4 and 7 hours.

Optionally, after the incubation step cells in the obtained bulk starter culture can be recovered/harvested/isolated/separated from the growth medium. Methods to recover/harvest/isolate/separate microbial cells from growth media are well known to the man skilled in the art and include centrifugation, (ultra)filtration or combinations thereof to name just a few. After recovering/harvesting/isolating/separating the cells by centrifugation and/or (ultra)filtration, they can be packaged for subsequent use or storage. The cells in the obtained mixed bulk starter cultures can also be aliquoted for subsequent use or storage without previously being recovered/harvested/isolated/separated.

The growth medium can be any growth medium suitable for the strains to be propagated. It can be a synthetic growth medium (ready-to-use liquid or powdered medium that first has to be reconstituted), whey or milk, to name just a few. In a preferred embodiment the growth medium is milk. “Milk” as used herein includes, but is not limited to, cow milk, human milk, buffalo milk, yak milk, horse milk, zebra milk, camel milk, donkey milk, reindeer milk, goat milk and sheep milk. In a preferred embodiment the milk is cow milk. “Milk” as used herein can be whole milk (about 3.5% (w/w) butterfat), skimmed milk (less than 0.5% (w/w) butterfat), semi-skimmed milk (1.5-1.8% (w/w) butterfat), low fat milk (about 1% (w/w) butterfat), reduced fat milk (about 2% (w/w) butterfat), milk protein concentrate comprising 6-17% (w/w) dry milk solids, or any other kind of milk. It can be pasteurized or non-pasteurized and/or concentrated (for example milk concentrated by UF) or non-concentrated milk and/or native or reconstituted milk.

The growth medium can be heat treated before the microbial cells are introduced into the medium. Suitable heat treatments include, but are not limited to, pasteurization methods such as high temperature/short time (HTST) pasteurization (heating medium, e.g. milk, to a temperature of 71° C. to 75° C., for about 15 to 30 seconds); ultra-high temperature (UHT) pasteurization (heating medium, e.g. milk, to a temperature of about 135° C. or higher for around 1-2 seconds); batch or vat pasteurization (heating large batches of medium, e.g. milk, to temperature of typically 63° C. for 30 minutes, followed by quick cooling to about 4° C.); higher heat/shorter time (HHST) pasteurization (pasteurization of medium, e.g. milk, that lies somewhere between HTST and UHT in terms of time and temperature). After the heat treatment the temperature of the growth medium should be adjusted to the temperature suitable for propagation of the cells.

In an embodiment the incubation step is performed under temperature and/or pH control. The pH control can be internally and/or externally, with external pH control being preferred. Internal pH control includes, but is not limited to, the use of buffering agents. External pH control includes, but is not limited to, addition of aqueous ammonia, sodium hydroxide or other food grade caustic. The pH is controlled at a pH of about 5 or higher, preferably between 5.0 and 6.5. More preferably, the pH is controlled between 5.5 and 6.5 and most preferred between 5.8 and 6.0.

The preparation of the bulk starter culture is done in a starter inoculation tank or vessel. Preferably, the tank or vessel is sanitized, i.e. cleaned and treated with a bactericidal sanitizer solution such as e.g. sodium hypochlorite. In an embodiment the preparation of the bulk starter culture is performed in a large-scale fermentor comprising from 10 to 100,000 litre growth medium, preferably 100 to 10,000 litre growth medium.

A further aspect of the invention involves a method of making a fermented product, the method comprising the steps of using a formulated culture concentrate or a bulk starter obtained thereof and adding one of them to a substrate and allowing it to ferment the substrate to produce a fermented product. When the substrate is cheese milk, the (bulk) starter culture can be added to the substrate in an amount ranging from 0.1% (w/w) to 5% (w/w), preferably, 0.2% (w/w) to 2.0% (w/w). The amount added depends on the application, i.e. the type of cheese to be made. The addition of the (bulk) starter culture to the substrate can be done immediately after its preparation; however, the (bulk) starter culture can also be stored for e.g. 1 to 72 hours before being added to the substrate. Storage is preferably done under cooling to e.g. 4° C. In the addition step the substrate is incubated under conditions favourable to the metabolic activity of the microbial cells, e.g. lactic acid bacteria cells, in the (bulk) starter culture, so as to obtain the expected and desired fermented product. The invention also relates to a method for manufacturing a food or feed product comprising adding a (bulk) starter culture according to the invention to a food or feed product starting material and keeping the thus inoculated starting material under conditions where the microbial cells, e.g. lactic acid bacteria, in the (bulk) starter culture are metabolically active, i.e. have acidification activity.

The substrate can be a food substrate such as a soya bean substrate, a meat substrate, a substrate for a bakery, wine, beverage, fruit juice or vegetable product or a dairy substrate, e.g. milk. Typically, the fermented product is a dairy food product or dairy food-derived product, e.g. cheese, yoghurt, butter, quark, sour cream, matured cream, infant milk, cream dessert, ice cream, inoculated sweet milk, buttermilk, kefir, koumiss, milk beverage, fermented whey-based beverage, fermented milk or drinking yoghurt.

In yet another embodiment, the invention thus provides use of a formulated culture concentrate as described herein or a bulk starter culture obtainable as described herein for producing a fermented milk product. In a preferred embodiment, the fermented milk product is cheese or yoghurt.

The invention also provides a method for preparing a fermented milk product comprising adding a formulated culture concentrate of the invention or a bulk starter culture obtainable as described herein to milk and subjecting the milk to conditions so as to produce a fermented milk product. In a preferred embodiment, the fermented milk product is cheese or yoghurt.

In yet another embodiment, the invention provides use of glycerol and cellulose for preventing/avoiding/diminishing freezing of a liquid composition such as a liquid composition comprising micro-organisms such as lactic acid bacteria at temperatures below −40° C.

### EXAMPLES

To illustrate the invention, the following examples are provided. These examples are not intended to limit the scope of the invention.

### Example 1

### Freezing Profile of a Composition According to the Invention

The example shows the freezing profile of a culture concentrate formulated with a standard composition comprising glycerol and a culture concentrate formulated with a composition according to the invention when subjected to a temperature of −70° C.

The standard composition comprised about 64 wt % glycerol, about 33 wt % yeast extract, about 0.8 wt % iron sulphate and was supplemented with water to a percentage of 100. The composition according to the invention comprised about 63 wt % glycerol, about 29 wt % yeast extract, about 0.9 wt % iron sulphate, about 1.2 wt % cellulose, about 4.2 wt % fructose, about 0.2 wt % monosodium glutamate and was supplemented with ammonium hydroxide solution (about 29% w/v) to a percentage of 100.

Mesophilic lactic acid bacteria were grown in a suitable medium, centrifuged and the obtained concentrates were reconstituted with supernatant to obtain a concentrated culture concentrate comprising about 1×10¹¹ cfu/ml. Each composition was mixed with part of the culture concentrate at a 2:1 ratio (i.e. 2 parts composition versus 1 part culture concentrate). The pH of the formulations was 6.0.

Next, a bag was filled with 4 kg culture concentrate formulated in standard composition and a bag was filled with 4 kg culture concentrate formulated in the composition according to the invention and both bags were placed in a −70° C. freezer. The temperature of the compositions was measured during the cooling process.

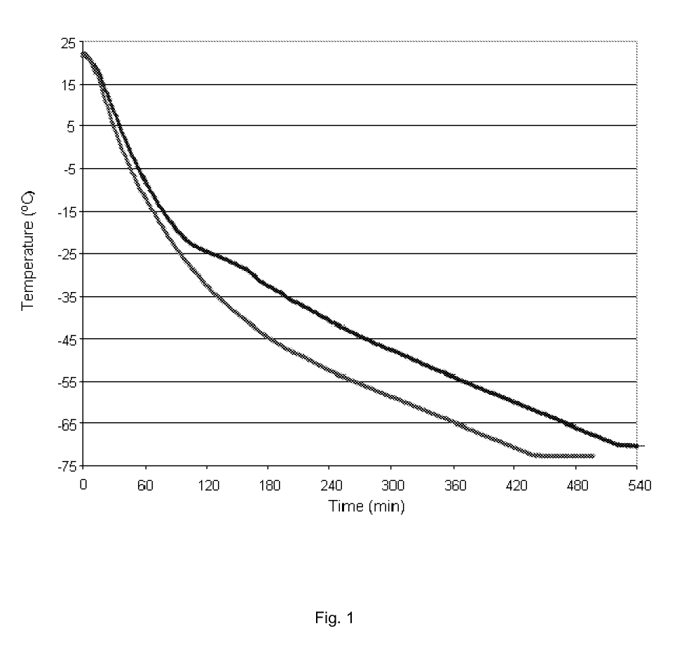

The results are shown in FIG. 1. The culture concentrate formulated in the composition according to the invention demonstrated a continuous temperature decrease, while the culture concentrate formulated in the standard composition showed a plateau at about −23° C., indicating that it goes through a phase change (e.g. freezing/ice formation) at that temperature. FIG. 1 clearly shows that the culture concentrate formulated in the composition according to the invention does not go through a phase change during cooling to a temperature of about −70° C. FIG. 1 further shows that the overall cooling rate is faster for the culture concentrate formulated in the composition according to the invention. Identical results were obtained when the compositions contained 48 wt % instead of about 64 wt % glycerol.

### Example 2

Stability and Acidification Activity of Culture Concentrates Formulated with Compositions According to the Invention or Standard Compositions when Subjected to Fluctuations in Temperature

The example shows the acidification activity of a culture concentrate formulated with a standard composition comprising glycerol and the acidification activity of a culture concentrate formulated with a composition according to the invention.

The standard composition comprised about 64 wt % glycerol, about 33 wt % yeast extract, about 0.8 wt % iron sulphate and was supplemented with water to a percentage of 100. The composition according to the invention comprised about 63 wt % glycerol, about 29 wt % yeast extract, about 0.9 wt % iron sulphate, about 1.2 wt % cellulose, about 4.2 wt % fructose, about 0.2 wt % monosodium glutamate, about 0.2 wt % silicone defoamer and was supplemented with ammonium hydroxide solution (about 29% w/v) to a percentage of 100.

Mesophilic lactic acid bacteria were grown in a suitable medium, centrifuged and the obtained concentrates were reconstituted with supernatant to obtain a concentrated culture concentrate comprising about 1×10¹¹ cfu/ml. The obtained culture concentrate was mixed at a 1:1 ratio with standard composition or at a 2:1 ratio with the composition according to the invention (i.e. 2 parts composition according to the invention versus 1 part culture concentrate), placed in plastic cups and placed directly in a −70° C. freezer. The pH of the formulations was 6.0.

The lactic acid bacteria were subjected to the following temperature fluctuations: 3 days −70° C.; 2 days −20° C.; 2 days −70° C.; 2 days −20° C.; and 1 day −70° C. Thereafter, they were removed from the freezer and tested for activity.

Just before measuring the activity of the lactic acid bacteria, the culture concentrate formulated with the standard composition was diluted 5× with cold RSM medium, while the culture concentrate formulated with the composition according to the invention was diluted 3.3× with cold RSM medium. This resulted in culture concentrates diluted 10×. The diluted culture concentrates were mixed until homogeneous and immediately tested for acid production.

Acidification activity was determined by inoculating cold 2%-fat UHT-pasteurised milk with 1.5% (v/v) or 3.0% (v/v) of the diluted cultures, incubating the inoculated milk for 2.5 hours at 31° C., and then measuring the change in pH of the milk versus non-inoculated milk.

Activity numbers shown in Table 1 represent the drop in the pH of the milk after 2.5 hours (ΔpH/2.5 hours) caused by acid production of the culture. The acidification activity of the freshly prepared culture was 1.47 for a 1.5% (v/v) inoculation and 1.82 for a 3% (v/v) inoculation. The results demonstrate that the acidification activity of lactic acid bacteria that have been subjected for ten days to several temperature fluctuations (between −20° C. and −70° C.) is higher when they are formulated with a composition according to the invention than when they are formulated with a standard composition. When formulated in a composition according to the invention there is no difference with the freshly prepared lactic acid bacteria. This shows that microorganisms are stable, even under extreme temperature conditions, when combined with the compositions according to the invention. The experiment was repeated with different strains. The strains showed a similar behaviour.

TABLE 1
Acidification activity of a lactic acid bacteria culture concentrate
formulated with a standard composition or a composition according
to the invention after being subjected to temperature fluctuations
during a time period of 10 days.

| Culture concentrate formulated | | Culture concentrate formulated with | |
|---|---|---|---|
| with standard composition | | composition according to the invention | |
| 1.5% (v/v) | 3.0% (v/v) | 1.5% (v/v) | 3.0% (v/v) |
| inoculation | inoculation | inoculation | inoculation |
| 1.10 | 1.52 | 1.47 | 1.76 |

### Example 3

### Stability and Acidification Activity of Culture Concentrates Formulated with Compositions According to the Invention or Standard Compositions after Prolonged Storage at −20° C.

The example shows the acidification activity of a culture concentrate formulated with a composition according to the invention after storage for five weeks at −20° C.

The composition according to the invention comprised about 68 wt % glycerol, about 29 wt % yeast extract, about 0.75 wt % iron sulphate, about 1.2 wt % cellulose, about 0.3 wt % silicone defoamer and was supplemented with ammonium hydroxide solution (about 29% w/v) to a percentage of 100. The pH of the composition was 6.0.

Two different Lactococcus lactis ssp. cremoris strains were grown in a suitable medium, centrifuged and the obtained concentrates were reconstituted with supernatant to obtain a concentrated culture concentrate comprising about 1×10¹¹ cfu/ml. The obtained culture concentrates were mixed at a 2:1 ratio with the composition according to the invention (i.e. 2 parts composition according to the invention versus 1 part culture concentrate), placed in plastic cups and placed directly in a −20° C. freezer. The lactic acid bacteria were stored for five weeks at −20° C. The acidification activity of the bacteria was determined after three and five weeks by removing them from the freezer and testing them for activity.

Just before measuring the activity of the lactic acid bacteria, the culture concentrate formulated with the composition according to the invention was diluted 3.3× with cold RSM medium. This resulted in a culture concentrate diluted 10×. The diluted culture concentrate was mixed until homogeneous and immediately tested for acid production.

Acidification activity was determined by inoculating cold 2%-fat UHT-pasteurised milk with 1.5% (v/v) or 3.0% (v/v) of the diluted culture, incubating the inoculated milk for 2.5 hours at 31° C., and then measuring the change in pH of the milk versus non-inoculated milk.

Activity numbers shown in Table 2 represent the drop in the pH of the milk after 2.5 hours (ΔpH/2.5 hours) caused by acid production of the culture. The results demonstrate that the acidification activity of lactic acid bacteria that have been formulated in the compositions according to the invention and that have been stored for five weeks at −20° C. do not loose their acidification activity compared to the formulated lactic acid bacteria that have not been stored. The results show that microorganisms formulated in the compositions according to the invention are stable, even when subjected to extreme temperature conditions for prolonged periods of time.

TABLE 2
Acidification activity of two Lactococcus lactis ssp. cremoris
strains that have been formulated with a composition according
to the invention after storage for five weeks at −20° C.

| Time at −20° C. | Strain A | | Strain B | |
|---|---|---|---|---|
| (weeks) | 1.5% (v/v) | 3.0% (v/v)) | 1.5% (v/v)) | 3.0% (v/v)) |
| 0 | 1.40 | 1.79 | 1.34 | 1.71 |
| 3 | 1.34 | 1.74 | 1.35 | 1.74 |
| 5 | 1.39 | 1.77 | 1.28 | 1.69 |

### Example 4

### Dosing of a Standard Composition and a Composition According to the Invention

The example shows the difference in dosing time from a standard composition and a composition according to the invention (both in a 4 kg bag) into a bulk start vessel.

The standard composition comprised about 64 wt % glycerol, about 33 wt % yeast extract, about 0.8 wt % iron sulphate and was supplemented with water to a percentage of 100. The composition according to the invention comprised about 67 wt % glycerol, about 30 wt % yeast extract, about 1.0 wt % iron gluconate, about 1.2 wt % cellulose, about 0.3 wt % silicone defoamer and was supplemented with ammonium hydroxide solution (about 29% w/v) to a percentage of 100.

Mesophilic lactic acid bacteria were grown in a suitable medium, centrifuged and the obtained concentrates were reconstituted with supernatant to obtain a concentrated culture concentrate comprising about 1×10¹¹ cfu/ml. Each composition was mixed with part of the culture concentrate at a 2:1 ratio (i.e. 2 parts composition versus 1 part culture concentrate). The pH of the formulations was 6.0.

Next, a bag was filled with 4 kg culture concentrate formulated in standard composition and a bag was filled with 4 kg culture concentrate formulated in the composition according to the invention and both bags were stored at −60° C.

The bag with standard composition was stored at −16° C. for 13 hours, thereafter at 6° C. and then emptied into a bulk starter vessel. The culture concentrate formulated in the standard composition was added to the bulk start vessel in about 45 minutes.

The bag with the composition according to the invention was stored at −20° C. for 8 hours and then emptied into a bulk starter vessel. It was emptied in a similar way as the bag with culture concentrate formulated in standard composition. The culture concentrate formulated in the composition according to the invention was added to the bulk start vessel in about 5 minutes.

### Example 5

### Stimulating Activity of Yeast Extract and Iron

In the example the stimulating activity of the compositions according to the invention in starter culture preparation is shown.

One litre of 2%-fat UHT milk was added to a previously sterilized 1200 ml fermentor vessel. Suspensions of yeast extract formulated with either iron pyrophosphate or iron sulfate were sterilized separately and added to the vessel to supply the milk with 0.02 wt % yeast extract and iron at a final amount of 2.4, 4.2 or 6 ppm.

Vessels were heated to 31° C., inoculated with freshly thawed culture concentrates and incubated for 7 hours under pH-control (pH between 5.9 and 6.1). After the incubation, the obtained starter culture was immediately cooled to a temperature below 8° C.

The obtained starter culture was tested for acidification activity by inoculating cold 2%-fat UHT-pasteurised milk with 3.0% (v/v) of the culture, incubating the inoculated milk for 2.5 hours at 31° C., and then measuring the change in pH of the milk versus non-inoculated milk. The experiment was done for five different mesophilic lactic acid bacteria strains. In the absence of iron the acidification activity of strain 1 and strain 2 was 1.18 and 1.44, respectively.

The results are shown in Table 3. They demonstrate that the compositions according to the invention stimulate lactic acid bacteria with respect to acidification activity. In concreto, the results show that the acidification activity increases when the final amount of iron added increases. This is independent of the source of iron. It is observed for iron sulphate as well as iron pyrophosphate. The increase is higher from 2.4 ppm to 4.2 ppm than from 4.2 ppm to 6.0 ppm. Therefore, it might be concluded that 4.2 ppm iron leads to near optimal stimulation. Ergo, iron should at least be added to a medium (e.g. milk) in an amount that provides a final concentration of 4.2 ppm iron in the medium.

Furthermore, addition of iron sulphate appears to be more effective than addition of iron pyrophosphate in increasing the acidification activity of lactic acid bacteria at low iron concentrations (e.g. a final iron concentration of 2.4 ppm), while no significant difference in acidification activity upon addition of iron sulphate or iron pyrophosphate can be observed at a final concentration of 4.2 and 6.0 ppm iron. This might be the consequence of increased bioavailability of iron sulphate compared to iron pyrophosphate due to better solubility and thus less precipitation in processing.

TABLE 3
Acidification activity (ΔpH/2.5 h at 31° C.) of mesophilic lactic
acid bacteria strains after addition of yeast extract and iron.

| | Iron sulphate | | | Iron pyrophosphate | | |
|---|---|---|---|---|---|---|
| | 2.4 ppm | 4.2 ppm | 6.0 ppm | 2.4 ppm | 4.2 ppm | 6.0 ppm |
| Strain 1 | 1.47 | nd | 1.53 | nd | nd | nd |
| Strain 2 | 1.77 | nd | 1.71 | nd | nd | nd |
| Strain 3 | 1.49 | 1.49 | 1.51 | 1.45 | 1.48 | 1.50 |
| Strain 4 | 1.54 | 1.56 | 1.57 | 1.48 | 1.56 | 1.58 |
| Strain 5 | 1.29 | 1.33 | 1.35 | 1.25 | 1.32 | 1.33 |

Nd means not determined

### Example 6

### Stimulating Activity of Yeast Extract and a Combination of Iron and Manganese

The example shows that mesophilic lactic acid bacteria strains which can be used as starter cultures in cheese production are stimulated by iron and manganese in growth and acid production.

Acid production was measured by inoculation of cold 2%-fat UHT-pasteurised milk with a known amount of the cultured strains, incubating the inoculated milk for 2.5 hours at 31° C., and then measuring the change in pH of the milk versus non-inoculated milk.

The strains were fermented in sterilized production medium in a standard fermentation vessel. The fermentation was carried out by maintaining the pH between 6-6.5 with ammonia at a temperature of about 30° C. The culture was harvested after ammonia consumption was stabilized. The culture was cooled to a temperature of below 10° C. and centrifuged. A concentration factor (harvested broth over pellet volume) of 15-20 was obtained. The obtained concentrate was used for further testing, e.g. with the cell concentrates a series of bulk starter culture fermentations was performed.

The obtained concentrate was added to UHT milk with and without iron sulphate and manganese sulphate to prepare a bulk starter culture. The final amount of iron and manganese in the milk was 8 ppm and 8 ppm, respectively. The bulk starter culture was prepared by fermenting the milk during 6 hours at 30° C. under pH-stat conditions. Ammonia was used to maintain the pH at 6.0-6.2. Samples (1.5 ml) of the cultures were taken after 5 and 6 hours fermentation. The samples were tested to establish the acidification activity of the culture under standard conditions. The samples were added to 100 ml UHT milk and the acidification activity was measured as described above.

The acidification activity of bulk starter cultures prepared by two different mesophilic lactic acid bacteria strains, either alone or in combination, after 5 and 6 hours fermentation was determined. A first bulk starter culture was prepared by inoculating UHT milk with a first strain in an amount of 0.06 wt %. A second bulk starter culture was prepared by inoculating UHT milk with a second strain in an amount of 0.10 wt %. A third bulk starter culture was prepared by inoculating UHT milk with the first strain in an amount of 0.03 wt % and the second strain in an amount of 0.05 wt %. The fermentation was performed as described above.

The results are reported in Table 4. Table 4 shows that the acidification activity of bulk starter cultures grown in the presence of iron and manganese is significantly increased. Table 4 also shows that this effect is seen in bulk starter cultures comprising a combination of strains. Fifteen further bulk starter cultures comprising combinations of two different strains were tested. All combinations showed an increase in acidification activity similar to that observed for the combination of strain 6 and 7 in the presence of 8 ppm iron and 8 ppm manganese (data not shown).

TABLE 4
Acidification activity of bulk starter cultures grown in milk with
and without iron and manganese (ΔpH/2.5 h at 31° C.).

| | | Strains | | |
|---|---|---|---|---|
| | Fermentation | | | Strain 6 + |
| Medium | time (hours) | Strain 6 | Strain 7 | Strain 7 |
| UHT milk | 5 | 1.11 | 1.20 | 1.14 |
| | 6 | 1.18 | 1.44 | 1.29 |
| UHT milk with 8 ppm | 5 | 1.52 | 1.33 | 1.40 |
| Fe + 8 ppm Mn | 6 | 1.52 | 1.64 | 1.61 |

In a further experiment, bulk starter cultures of strain 6 were prepared in UHT milk with and without iron sulphate and manganese sulphate. Several different final iron and manganese concentrations were used in this experiment. The concentrations tested are indicated in Table 5. The bulk starter culture was prepared as described above with the proviso that the UHT milk was inoculated with 0.6 wt % cell concentrate. The acidification power was measured as described above with the proviso that 6 gram sample was taken from the cultures after 7 hours and added to 200 g UHT milk.

The results are shown in Table 5 and demonstrate that the acidification activity of the bulk starter cultures prepared in milk with different amounts of iron and manganese is increased compared to acidification activity of the bulk starter cultures prepared without iron and manganese supplementation.

TABLE 5
Acidification activity of bulk starter cultures
grown in milk with and without different iron and
manganese concentrations (ΔpH/2.5 h at 31° C.).

Fe

| M | 0 ppm | 2.4 ppm | 4.8 ppm | 7.2 ppm | 9.6 ppm |
|---|---|---|---|---|---|
| 0 ppm | 1.61 | 1.67 | 1.66 | 1.66 | 1.66 |
| 2.4 ppm | 1.65 | 1.67 | nd | nd | nd |
| 4.8 ppm | nd | nd | nd | nd | nd |
| 9.6 ppm | nd | nd | nd | 1.72 | nd |

Nd means not determined

### Example 7

### Stimulating Activity of Yeast Extract and a Combination of Iron, Manganese and Molybdate

The example shows that mesophilic lactic acid bacteria strains are stimulated by a composition comprising yeast extract, iron sulphate, manganese sulphate, sodium molybdate or a combination thereof. The final amount of yeast extract, iron, manganese and molybdate in the milk was 0.02 wt %, 8 ppm, 8 ppm and 8 ppm, respectively.

The strains were fermented as described in Example 6. The bulk starter cultures were prepared as described in Example 6 with the proviso that the UHT milk was inoculated with 0.6 wt % cell concentrate. Acid production was measured as described in Example 6 with the proviso that 6 gram sample was taken from the cultures after 5 and 6 hours and added to 200 g UHT milk. The experiment was done with two different strains.

The results are shown in Table 6 and demonstrate that the acidification activity of the bulk starter cultures prepared in milk with yeast extract, iron and manganese, or molybdate is increased compared to acidification activity of the bulk starter cultures prepared without any supplementation. It is further observed that the highest increase in acidification activity is found when Fe and Mn are present.

TABLE 6
Acidification activity of bulk starter cultures grown
in milk with and without yeast extract, iron, manganese
and/or molybdate (ΔpH/2.5 h at 31° C.).

| | Strain 8 | | Strain 9 | |
|---|---|---|---|---|
| Addition | 5 hours | 6 hours | 5 hours | 6 hours |
| No addition | 0.64 | 1.08 | 0.33 | 0.43 |
| YE | 0.74 | 1.08 | 0.38 | 0.58 |
| Fe + Mn | 0.93 | 1.23 | 0.79 | 1.07 |
| Mo | 0.73 | 1.04 | 0.55 | 0.79 |
| YE + Fe + Mn | 1.07 | 1.27 | 0.79 | 1.07 |
| YE + Mo | 0.67 | 1.08 | 0.63 | 0.85 |
| Mo + Fe + Mn | 0.85 | 1.25 | 0.75 | 1.07 |
| YE + Fe + Mn + Mo | 1.04 | 1.27 | 0.79 | 1.07 |

1. A micro-organism activating and/or a micro-organism stabilising composition comprising:
yeast extract,
a polysaccharide, and
a cryoprotectant.

2. A composition according to claim 1, further comprising iron, manganese, molybdenum or zinc salts or any combination thereof.

3. A composition according to claim 1, characterized in that the composition comprises:
5-45 wt % yeast extract,
0.5-2.5 wt % polysaccharide, and
20-80 wt % cryoprotectant,
wherein the total percentage of the compounds is 100.

4. A composition according to claim 3, further comprising 0.1-10 wt % iron, manganese, molybdenum or zinc salts or any combination thereof and wherein the total percentage of the compounds is 100.

5. A composition according to claim 1, characterized in that the composition is liquid at a temperature of −60° C. to −20° C.

6. A method for preparing a formulated culture concentrate comprising combining a composition according to claim 1 with a culture concentrate, optionally mixing the compounds and/or packaging the formulated culture concentrate and/or lowering the temperature to −80° C. or to −20° C.

7. A method according to claim 6, characterized in that the culture concentrate comprises lactic acid bacteria.

8. A formulated culture concentrate comprising a composition according to claim 1 and a (starter) culture concentrate.

9. A formulated culture concentrate comprising:
2.5-39 wt % yeast extract,
0.25-2.2 wt % polysaccharide,
10-70% cryoprotectant, and
15-70% culture concentrate,
wherein the total percentage of the compounds is 100.

10. A formulated culture concentrate according to claim 9, further comprising 0.1-10 wt % iron, manganese, molybdenum or zinc salts or any combination thereof and wherein the total percentage of the compounds is 100.

11. A formulated culture concentrate according to claim 9, characterized in that the composition is liquid at a temperature of −60° C. to −20° C.

12. Use of a composition according to claim 1 for activating and/or stabilizing a micro-organism.

13. A method for preparing a bulk starter culture comprising adding a formulated culture concentrate according to claim 9 to a suitable medium and propagating the culture to a desired density.

14. Use of a formulated culture concentrate according to claim 9 for producing a fermented milk product.

15. A method for preparing a fermented milk product comprising adding a formulated culture concentrate according to claim 9 to milk and subjecting the milk to conditions so as to produce a fermented milk product.
